(19) United States
(12) Patent Application Publication
(10) Pub. No.: US 2019/0030279 A1
(43) Pub. Date: Jan. 31, 2019

(54) Method of Conditioning a Living Body to Respond Appropriately to Stimuli

(71) Applicant: Dawn Nowlin, Leesburg, VA (US)

(72) Inventor: Dawn Nowlin, Leesburg, VA (US)

(21) Appl. No.: 16/132,279

(22) Filed: Sep. 14, 2018

Related U.S. Application Data
(63) Continuation-in-part of application No. 14/996,202, filed on Jan. 14, 2016, now abandoned.
Application No. 14/996,202 is a continuation-in-part of application No. 14/004,257, filed on Sep. 10, 2013, now abandoned.
Application No. 14/004,257 is a continuation-in-part of application No. 13/044,986, filed on Mar. 10, 2011, now abandoned.
Application No. 13/044,986 is a continuation-in-part of application No. 12/235,360, filed on Sep. 22, 2008, now abandoned.

(51) Int. Cl. A61M 21/02; A61N 1/36; A61H 23/02; A61H 23/06; A61N 1/04

(52) U.S. Cl. CPC A61M 21/02; A61N 1/36025; A61M 2021/0027; A61H 23/06; A61N 1/0456; A61H 23/02

(57) ABSTRACT

A method of conditioning a living body of a patient to associate the unconditioned stimulus (US) of decreased sympathetic nerve activity (SNA) with the conditioned stimulus (CS) or offending agent to cease or reduce defensive reactions or symptoms. The US involves non-invasive, mechanical stimulation of the sympathetic ganglia, which is paired with the offending stimuli or representation of the offending stimuli to modify pathologically conditioned reflexes of various systems involved in a reaction. In addition, conditions are treated by using digital representations, preferably provided via a computer, to represent the offending stimulus in order to engage the multimodal functioning of the brain. Sensory stimulation is used in conjunction with the various modalities of stimuli to condition the body to respond more appropriately to the stimulus.

### CONTINUITY

This application is a continuation-in-part application of U.S. patent application Ser. No. 14/996,202, filed on Jan. 14, 2016, which is a continuation-in-part application of U.S. patent application Ser. No. 14/004,257 filed on Sep. 10, 2013, which is a continuation-in-part application of continuation-in-part application Ser. No. 13/044,986, filed on Mar. 3, 2011 to non-provisional patent application Ser. No. 12/235,360, filed on Sep. 22, 2008, and priority is claimed thereto.

### FIELD OF THE PRESENT INVENTION

The present invention is a method of conditioning a living body of a patient to associate a decrease in sympathetic nerve activity from stimulation of the sympathetic ganglia as the Unconditioned Stimulus with a secondary stimulus known to cause physiological reactions of bodily systems; to ultimately reduce reactive behavior to such stimuli. In addition, the method of the present invention takes advantage of multimodal functioning of the brain, thus using multiple modes of stimuli as the secondary stimulus.

### BACKGROUND OF THE PRESENT INVENTION

### Pathological Reactions to Stimuli

A broad range of physiological reactions may be initiated by stimuli from the external environment. Some reactions may be pathological. Examples include headaches from fluorescent light, skin rashes from heat or acid reflux to specific foods. Such reactions are idiosyncratic to particular individuals and not generalized, such as reactions to poison ivy, for example, which affects a large majority of the population. Conditions which are caused by pathological reactions to stimuli are recognized as “sensitivities” or “hypersensitivies” and remain an understudied field in medical science. The phenomenon of sensitivities is defined by an increase in the speed and severity of a reaction whereas habituation is a decrease in the speed and severity. If immune involvement is ruled out, such conditions are viewed as unrelated and are investigated within specialized domains of medicine (gastroenterology, dermatology, etc.). Reactions to harmless stimuli have been primarily recognized within the field of allergy and immunology with the reactions being attributed to aberrant immune activity. However, the inventor has discovered that inappropriate reactions or overreactions to harmless stimuli may occur with other systems in the body and may or may not involve the immune system. The standard medical treatment for most of these types of conditions is avoidance or the use of medications to mitigate the symptoms.

A significant number of reactions to harmless stimuli do not involve the immune system. Increasingly, evidence is showing that mast cells, which play a central role in the allergic response, can be activated directly, by non-allergic triggers. Such triggers may include environmental chemicals, foods, drugs, odors, temperature changes, physical or emotional stimuli and stress. Mast cells are found in tissues throughout the body and it is well established that there is an anatomic association between mast cells and nerves in most tissues. The nervous system plays a central role in sensitivities. The symptoms can be as pronounced and can even be as severe as those of allergies, with no immune involvement. It has only been recently recognized that sensitivity-related illness (SRI) may involve various organ systems and evoke wide ranging physical or neuropsychological manifestations. The inventor has concluded that a particular bodily system's reaction to a substance or stimulus may be due to an association being established between a stimulus and increased sympathetic nerve activity. A stimulus associated with increased sympathetic nerve activity may result in a broad range of symptoms depending on the system or systems involved, including the immune system. However, this invention is not based on treating the immune system, but instead approaches the physiological reactions to stimuli by addressing a pathologically conditioned reflex which has also been shown to be a contributor to the development of allergies. The invention takes advantage of adaptive mechanisms of the body, inherent in stimulus-response behavior, utilizing a conditioning method that enables the bodily system to respond to a stimulus in a normal and healthy manner. The method is not intended to treat cases of anaphylaxis.

The first known study on the phenomenon of conditioning was in 1902, by Ivan Pavlov, demonstrating that conditioned reflexes may be learned by associating a conditioned stimulus (CS) with an unconditioned stimulus (US). Pavlov concluded that a connection was made in the nervous system linking an external stimulus to an unconditioned reflex, transforming the reflex into a conditioned reflex, activated by an external stimulus. This invention utilizes a non-invasive, mechanical stimulus to the sympathetic ganglia as the primary stimulus, which activates an unconditioned reflex of decreased Sympathetic Nerve Activity (SNA), coupled with a stimulus identified to be causing the physiological reactions. The CS presented may be the actual substance or stimulus, contextual linguistic audio digital signals or visual representations of the stimulus to create a new association of the treated stimulus. The stimulation to the sympathetic nervous system and use of various representations of offending stimuli can be integrated and, in fact, are connected through a temporal pairing.

### The Immune System and Classical Conditioning

Until relatively recently, the immune system had been regarded as an autonomous host defense system that evolved for the primary purpose of identifying and destroying pathogenic microbes and assisting in tissue repair. However, in the last few decades extensive research, particularly in the field of psychoneuroimmunology, has revealed a strong, integrative relationship between the central nervous system (CNS) and the immune system. The historical stance on the autonomy of the immune system was due to the fact that neuroscience and immunology developed independently for many years, which contributed to the lack of understanding about how the brain communicates with the immune system. See Elenkov, Ilia J., Ronald L. Wilder, George P. Chrousos, and E. S. Vizi 2000. The sympathetic nerve—an integrative interface between two supersystems: the brain and the immune system. Pharmacological reviews 52(4): 595-638. Also, see: Solomon, George F. 1987. “Psychoneuroimmunology: interactions between central nervous system and immune system.” Journal of Neuroscience Research, 18(1): 1-9. Ziemssen, Tjalf, and Simone Kern. 2007. “Psychoneuroimmunology—cross-talk between the immune and nervous systems.” Journal of neurology, 254(2): 118-1111

There are two pathways that link the brain and the immune system, allowing for bi-directional communication. The hypothalamic-pituitary-adrenal (HPA) axis provides a neuroendocrine pathway, and the autonomic nervous system (ANS) provides a direct neural link. The method used in this invention modulates communication between the Sympathetic Nervous System (SNS) and the brain, an important physiological link in the phenomenon of classical conditioning which can affect multiple systems as well as the immune system. Studies have shown that altered immune function can be induced through classical behavioral conditioning (Elenkov et al. 2000; Ader et al. 1987; Metalnikov 1926). Stimuli in the environment may become randomly associated with stress or symptoms from existing conditions which are experienced at the time of exposure. This coupling of the two stimuli is learned and stored, and may cause a chronic pattern of reactivity. See Elenkov, I. J., R. L. Wilder, G. P. Chrousos and E. S. Vizi (2000). “The Sympathetic Nerve—An Integrative Interface between Two Supersystems: The Brain and the Immune System.” Pharmacological Reviews 52(4): 595-638. Also see Ader R, Grota L J, Cohen N. (1987). Conditioning phenomena and immune function. Ann N Y Acad Sci. 1987; 496:532-44.

A series of studies in the 1920's (Metal'nikov et al 1926) provided strong evidence that immune reactions could be conditioned by classical Pavlovian means. The studies demonstrated that conditional reflexes can play a very important role not only in the immune response but also in a broad range of ailments. Experiments were conducted on laboratory animals to measure various reactions to conditioned stimuli. B. anthracoides or staphylococcus filtrates were injected into the peritoneal cavity and conditioned with a scratch to the skin or a heated metal plate placed on the skin. Animals that were conditioned to the skin scratch or heated metal plate showed an immediate increase in leukocytes in the peritoneal cavity from the skin stimulus alone, demonstrating that random stimuli can be conditioned to elicit the same defense behavior when introduced alone, with no infectious agents present. The authors concluded that this phenomenon was likely ubiquitous and that everything surrounding the patient may act as conditional stimuli, perpetuating the illness when the original cause is no longer present. This conclusion suggests that the types of potential CS are wide-ranging. In the study, the conditioned stimuli were both sensory stimuli that produced specific skin sensations that became paired with the injection of the bacteria. However, conditional stimuli can be derived from any of the senses. Once a stimulus is conditioned, a bodily system (including the immune system) may continue with pathological behavior due the association that was established. This led the authors to conclude that many chronic and nervous diseases such as asthma, heart disorders, or other neuroses occur under the influence of conditional stimuli. In other words, random stimuli can become conditioned due to the presence of an illness or other stressful conditions that involve changes in sympathetic nerve activity.

The inventor has discovered that the human body can react inappropriately to a vast number of benign substances or stimuli caused by conditioned reflexes. Conditioned stimuli may include naturally-occurring substances in the external environment but also non-physical stimuli such as natural language (Vinogradova and Sokolov 1957) or visual symbols (Icenhour et al, 2015). Recent investigations by Icenhour et al (2015) demonstrate that pain-related fear learning through classical conditioning could link visual symbols to symptoms of Irritable Bowel Syndrome (IBS). For the fear acquisition, predictive visual cues of images consisting of circles or squares (CS) were paired with artificially-induced intestinal pain. It was hypothesized that associative learning processes may contribute to pain-related anticipatory fear due to associations of the pain experience with internal or external cues. Presentation of the visual cues later reproduced similar intestinal symptoms. Subjects with IBS responded with enhanced reinstatement from the visual cues compared to subjects who underwent conditioning with no history if IBS. In other words, the response to the predictive cues (circle or square images) was more easily reestablished in IBS patients, suggesting that earlier conditioning may be contributing to the chronicity of the condition or that IBS patients are more prone to conditioning related to the intestines. These findings provide further evidence that cognitive processes are not separate from the immune system or other physiological systems and can be involved in pathological conditioning as well as corrective conditioning processes. This invention's method utilizes the same learning phenomenon to reverse the conditioned reflex by pairing the CS with decreased sympathetic nerve activity as opposed to the increased sympathetic nerve activity which is universally present during conditions of stress, (regardless of the type of stress) and has also been shown to be active in negative emotions such as fear and anger. See Metal'nikov S, Chorine V (1926) Role des reflexes conditionnels dans l'immunité. Ann Inst Pasteur Paris 40:893-900. Also see Icenhour, A. L., J.; Benson, S.; Schlamann, M.; Hampel, S.; Engler, H.; Forsting, M.; Elsenbruch, S. (2015). “Neural circuitry of abdominal pain-related fear learning and reinstatement in irritable bowel syndrome.” Neurogastroenterol Motil 27: 114-127. Also see Sokolov, Evgeny. N. and Olga S. Vinogradova 1975. Neuronal Mechanisms of the Orienting Reflex. Hillsdale, N.J.: Lawrence Erlbaum Associates.

### Unconditioned Stimulus Used in Method: Decreased Sympathetic Nervous Activity

The traditional view of an autonomous immune system has been successfully challenged over the last few decades. There is now strong evidence that the brain and the immune system, which are the two major adaptive systems of the body, maintain a bidirectional flow of messages and that there is direct sympathetic innervation of immune organs. The primary pathway for the neural regulation of immune function is provided by the sympathetic nervous system (Elenkov at al 2000; Nance et al 2007). The autonomic nervous system (ANS) regulates the functions of all innervated tissues and organs and is not under conscious control. See Elenkov, I. J., R. L. Wilder, G. P. Chrousos and E. S. Vizi. 2000. “The Sympathetic Nerve—An Integrative Interface between Two Supersystems: The Brain and the Immune System.” Pharmacological Reviews 52(4): 595-638. Also see Nance, D M, Sanders, V M. 2007. Autonomic Innervation and Regulation of the Immune System (1987-2007). Brain Behav Immun. 2007 August; 21(6): 736-745. Additionally, see Stein C, Dal Lago R, Fferreir J B, Casali K R, Della Mea Plentz R. 2011. Transcutaneous electrical nerve stimulation at different frequencies on heart rate variability in healthy subjects. Autonomic Neuroscience: Basic and Clinical 165 (2011) 205-208. Also see: Ziemssen, Tjalf, and Simone Kern. 2007. “Psychoneuroimmunology-cross-talk between the immune and nervous systems.” Journal of Neurology, 254(2): 118-1111., Abboud, Francois M., Sailesh C. Harwani, and Mark W. Chapleau. 2012. “Autonomic neural regulation of the immune system. Implications for hypertension and cardiovascular disease.” Hypertension, 59(4): 755-62. A number of recent studies and patent claims have shown that modulation of regional SNA can successfully treat a number of unrelated conditions by targeting a specific organ system (Barbut, Rozenberg, and Heinimann 2013; Rezai 2005; Poon et al. 2014; Libbus and Moffitt 2015). These more invasive methods involve surgical procedures that implant electrical devices contacting either the sympathetic ganglia (at the level of the affected organ) or the vagus nerve in the neck to treat a broad range of pathology. Table 1 shows a list of conditions that are treated with these methods. The range of conditions is notable since the approach is basic (modulation of SNA) and the same for all of the conditions and suggests that sympathetic nerve activity plays a key role in the pathology of these conditions.

TABLE 1
Conditions responding to treatment with methods using stimulation
to the sympathetic nervous system or vagus nerve.

| | | |
|---|---|---|
| Headaches | Alzheimer's | Pain syndromes |
| Migraines | Autism | Fatigue |
| Cardiac disorders | Bulimia | Food sensitivities |
| Excessive blushing | Chronic heart failure | Anxiety |
| Hypertension | Memory disorders | Dyspepsia |
| Renal disease | Mood disorders | Gastroparesis |
| Heart failure | Cancer | Esophageal reflux |
| Angina | Poor blood circulation | Ulcerative colitis |
| Intestinal motility | Leaky gut | Anorexia |
| disorder | Severe mental diseases | Fertility |
| Biliary disorders | Inflammation | Obsessive Compulsive |
| Bronchial disorders | Bipolar disorder | Disorder |
| Tracheal disorders | Obesity | Fibromyalgia |
| Pulmonary disorders | Hyperhidrosis | Tinnitus |
| Epilepsy | Attention deficit | Alcohol addiction |
| Hard-to-treat depression | hyperactivity disorder | Diabetes |
| Multiple sclerosis | (ADHD) | Dysautonomia |

Historically, the sympathetic nervous system has been considered to be a system that responds universally to stimuli with a fight or flight response. However, recent studies have shown that sympathetic nerve activity may also respond differentially with target organs and can occur acutely at varying magnitudes and in opposing directions, contributing differentially to various disease states. The sympathetic nervous system has been shown to exhibit differential control of sympathetic outflow. Responses to different stimuli provoke changes selectively in regional sympathetic nerve activity. In other words, specific stimuli can cause increased SNA in particular regions, resulting in reactions from corresponding target organs. These recent findings suggest that stimulus-response behavior involving the sympathetic nervous system may be contributing to a wide variety of conditions. The inventor discovered that mechanically disrupting sympathetic nerve activity at the sympathetic ganglia while simultaneously presenting a stimulus known to elicit physiological reactions could modulate behavior of the target organs. See: (Guild et al. 2007; Rahmouni 2007; Osborn and Fink 2010; Subramanian and Mueller 2016).

### Mechanical Stimulation of Sympathetic Chain

Studies have shown that non-invasive, transcutaneous electrical nerve stimulation (TENS) of the sympathetic chain decreases activity of the sympathetic nervous system (Stein et al. 2011). The inventor discovered that mechanical stimulation in the same area of the sympathetic ganglia along the spine produces similar results, providing the unconditioned stimulus necessary for this invention's method to reverse pathologically conditioned reflexes. Mechanical stimulation is based on methods derived from the emerging new subfield of mechanobiology, a domain of medical research that utilizes physical forces for therapeutic purposes (Huang et al. 2013). In this field of medicine, force or stimulation is being used on cells, molecules and tissues. It has been used to address the level of transient receptor potential channels (cell targeting) and intracellular mechanosignaling pathways (molecule targeting) and mechanotransduction. According to Huang, the effectiveness of the mechanical therapies currently in clinical use demonstrates the importance of physical forces in physiological control. Cells are apparently mechanosensitive and collectively cells respond to perturbations to their local mechanical environment resulting in tissue-level observations (Kung 2005). Mechanical stimulation of the sympathetic chain is preferable over surgical insertion of electrical devices for direct stimulation of the nerve and is the least invasive method to disrupt sympathetic nerve activity. Mechanical stimulation allows for a non-pharmacological, non-invasive approach to reduce activity of the sympathetic nervous system.

### Conditioned Stimulus Used in Method: Multimodal Stimulus Input

Random stimuli in the environment can be conditioned due to the presence of stress or illness, therefore the types of stimuli that can be involved are as varied as objects and stimuli in any external environment. In other words, the number of stimuli potentially involved in pathological conditioning is vast. Studies have shown that neural circuits in certain regions of the brain display common patterns of activation for concrete objects regardless of whether the objects are presented visually, auditorily or verbally (Simanova 2014; Marinkovic et al. 2003; Bright et al. 2004; Kircher et al. 2009; Price 2010). Decoding of semantic information is possible from different stimulus modalities, which also engages the symbolic level of cognitive processes. Thus, some cases of pathological conditioning can be addressed with digital signals created from a linguistic representation.

According to Pavlov, there exists only a first system of signals of reality in animals, allowing the brain to receive and analyze stimuli within the organism as well as outside the organism. In humans, there exists both this first level as well as a second level of signals: language or symbols. Words and symbols can function as stimuli in humans, and have been shown to mobilize humans just as a concrete stimulus. Words are symbols and abstractions and the conditioned stimulus can be generalizable. A linguistic representation may be converted to an audio digital signal and transmitted repeatedly during treatment. The invention engages unconscious processes by transmitting the digital signals at low decibels that are unavailable to conscious perception (subthreshold). Studies have shown that brain activity does index the acquisition of a conditional response to subthreshold stimuli and there is strong evidence for associative learning outside awareness in central nervous system activity (Wong et al 1991, Wong et al 2004). The digital audio signal is transmitted via speaker embedded in a cuff or mat on which the patient lies.

By presenting the offending stimulus in one of the three stimulus modalities and simultaneously introducing a sensory stimulus that decreases activity of the sympathetic nervous system, an association is established between the unconditioned stimulus and the benign stimulus altering the reaction. The inventor has found that the stimulus being treated may be introduced to the subject in various modalities, including an audio digital representation of the stimulus provided by a computer or other device. The multimodal processes of the brain allows for a semantic interpretation of the stimulus. The inventor has found that cognitive processes can interpret the representation of the stimulus used in treatment to allow for a pairing of the two stimuli for the conditioned effect. The use of audio digital representations is not applicable in all cases involving pathological conditioning.

See Simanova I, Hagoort P, Oostenveld R, van Gerven M A. 2014. Modality-independent decoding of semantic information from the human brain. Cereb Cortex. 2014 February; 24(2):426-34. See also Bright P, Moss H, Tyler I. K. 2004. Unitary vs multiple semantics; PET studies of word and picture processing. Brain Lang. 89:417-432. See as well Markinovic K, Dhond R P, Dale A M, Glessner M, Can V, Halgren E. 2003. Spatiotemporal dynamics of modality-specific and supramodal word processing. Neuron. 38:487-497. See Kircher T, Sass K, Sachs O, Krach S. 2009. Priming words with pictures: neural correlates of semantic associations in a cross-modal priming task using fMRI. Hum Brain Mapp. 30:4116-4128. See additionally Wong P S, Bernat E, Bunce S, Shevrin H. 1991. Brain indices of nonconscious associative learning. Conscious Cogn. 1991 December; 6(4):519-44. Also see Wong P S, Bernat E, Snodgrass M, Shevrin H. 2004. Event-related brain correlates of associative learning without awareness. Int J Psychophysiol. 2004 August; 53(3):217-31.

### SUMMARY OF THE PRESENT INVENTION

The method of the present invention is preferably enacted as follows:

The subject is treated by presenting the offending stimulus to the body (to which the body is inappropriately reacting), via one of the stimulus modalities, including (but not limited to) a computer playing a digitized sound, displaying a visual stimulus on a computer monitor, or introducing the actual substance or stimulus to the surface of the skin. Electrical stimulation from TENS or mechanical stimulation is administered at the location of the sympathetic ganglia. Stimulation of these areas has been shown to decrease activity of the sympathetic nervous system. The simultaneous stimulation of sympathetic ganglia with the input of the stimulus signal forms a new association with the stimulus thereby modifying the physiological reaction. The present invention is designed to engage the multimodal functioning of the brain through audio signals, visual signals or presentation of a stimulus to the surface of the skin.

The mechanism underlying the effect of the treatment is a conditioned association whereby the stimulus is “coupled” with decreased sympathetic nerve activity of the stimulus. The modified association with the conditioned stimulus alters the behavior of the effected system(s). The therapeutic mechanical stimulation used in conjunction with the presentation of the stimulus decouples the harmless stimulus with increased sympathetic nerve activity. The presentation of the conditioned stimulus in any mode has no therapeutic value. It is only the stimulation of the sympathetic ganglia chain in conjunction with the presentation of the conditioned stimulus that modulates the pathologically conditioned reflex.

The human body can inappropriately react to a vast number of substances or stimuli. Therefore, a database, maintained by a computer, is used to include a large number of stimuli organized in a hierarchical structure, including visual, digital contextual, or auditory modes depending on the stimulus.

Mechanical or electrical stimulation or a mat capable of providing sympathetic ganglia chain stimulation and the audible mode of stimulus presentation may be employed by the method of the present invention. The patient would preferably lay his or her back on the mat. The mat contains a speaker, which is preferably embedded or integrated into the mat, which contains vibratory motors designed for stimulation. Alternate embodiments of the present invention may employ a cuff equipped with a speaker and/or at least one vibration motor for use in lieu of a mat.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 depicts a flowchart detailing the method of the present invention as performed by a practitioner.

FIG. 2 exhibits a view of the mat employed in the method of the present invention, as seen from the top.

FIG. 3 shows a flow chart detailing the process of use of the software application of the present invention as used by a practitioner.

### DETAILED DESCRIPTION OF THE PREFERRED EMBODIMENTS OF THE PRESENT INVENTION

The present specification discloses one or more embodiments that incorporate the features of the invention. The disclosed embodiment(s) merely exemplify the invention. The scope of the invention is not limited to the disclosed embodiment(s). The invention is defined by the claims appended hereto.

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to effect such feature, structure, or characteristic in connection with other embodiments whether or not explicitly described.

The present invention is a method that serves to treat a specific stimulus or representation of the stimulus via various modalities in order to ultimately condition the body's natural physiology to accept such specific irritant that initially caused the sensitivity reaction. In one embodiment, stimulation is administered to locations near the sympathetic ganglia, along each side of the spine, with the use of vibratory motor stimulation. This stimulation is utilized to temporarily decrease activity of the sympathetic nervous system. Meanwhile, the stimulus or digital representation specific to the stimulus found to cause the irritation is presented. The combination of the presented stimulus and the stimulation of the sympathetic ganglia conditions the body to not react to that particular stimulus. In other words, the cutaneous exposure of an offending stimulus or different digital or visual signals, provided via a computer, that represent an irritant transmitted at the same time as decreasing sympathetic nerve activity ultimately conditions the various systems to respond without defensive or reactive behavior.

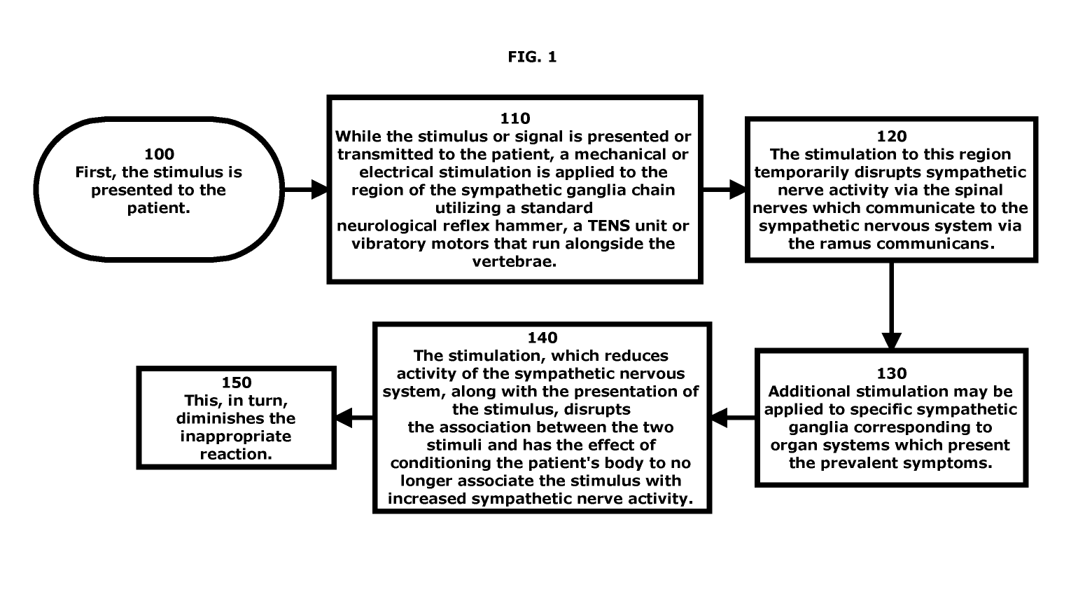

The method of the present invention, as depicted in FIG. 1, is preferably as follows: First, the stimulus is presented to the patient. (100) It should be noted that the patient also might be referred to as person or subject. In one embodiment, an offending stimulus is presented to the surface of the skin. In another embodiment, the stimulus may be presented via a digital audio signal transmitted, preferably via a computer. The signal being transmitted is representative of what the actual potential offending agent is purported to be. A digital audio signal means that the sound can be words representing the purported irritant. In another embodiment, the stimulus may be presented via a visual representation or cutaneous exposure.

While the stimulus or signal is presented or transmitted to the patient, a mechanical or electrical stimulation is applied to the region of the sympathetic ganglia chain utilizing a standard neurological reflex hammer, a TENS unit or vibratory motors that run alongside the vertebrae. (110) The stimulation to this region temporarily disrupts sympathetic nerve activity via the spinal nerves which communicate to the sympathetic nervous system via the ramus communicans. (120) Additional stimulation may be applied to specific sympathetic ganglia corresponding to organ systems which present the prevalent symptoms. (130) The stimulation, which reduces activity of the sympathetic nervous system, along with the presentation of the stimulus, disrupts the association between the two stimuli and has the effect of conditioning the patient's body to no longer associate the stimulus with increased sympathetic nerve activity. (140) This, in turn diminishes the inappropriate reaction. (150)

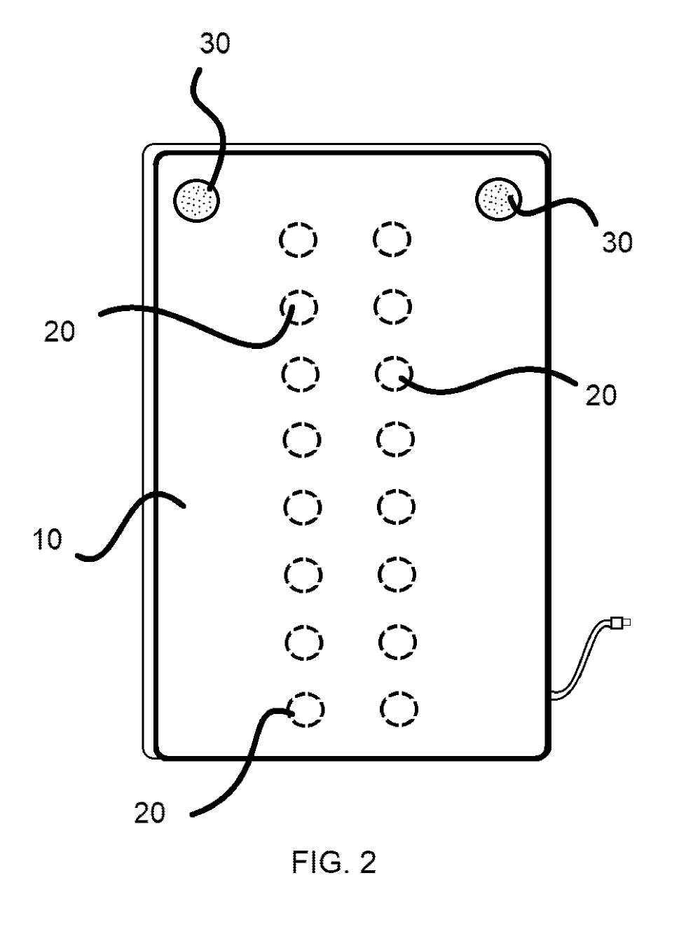

The mat that is utilized in a preferred embodiment of the present invention is preferably a thin foam mat that encases a series of vibratory motors as depicted in FIG. 2. The series of vibratory motors are, in one embodiment, arranged in two columns which are each 4 feet (121.92 cm) in length; there is a 3 inch (7.62 cm) separation between the two columns. The series of vibratory motors provides a gentle percussion when activated by the practitioner, via the software program. The mat is preferably placed on a treatment table and the patient lies on the mat so that the vibratory motors make contact at the locations medial to the paraspinal muscles, specifically between the spine and the paraspinal muscles of the patient. When activated, the device stimulates the sympathetic ganglia chain areas with a gentle vibratory motion. The vibratory stimulus occurs while the offending stimulus is presented to the patient, as described above. Therefore, it is preferred, for the audio representations of the stimulus, that at least one speaker (30) is housed within the mat (10), preferably disposed at a top of the mat (10) (near where the patient's head rests) which is configured to transmit the digital audio signal(s). Alternately, a cuff equipped with at least one speaker may be used in lieu of the mat (10). The speaker is preferably disposed within the cuff noted above, and the stimulation administered to the sympathetic ganglia by the doctor/practitioner would be manually applied if percussion is not provided by the mat. It should be understood that the mat (10) and the cuff are of conventional design. The present invention is designed to engage the multimodal functioning of the brain through audio signals or stimuli shown on the monitor of the computer from the software program or presentation of the actual substance or stimulus to the surface of the skin.

An overview of the mat apparatus and overarching system of the present invention and how it is used is outlined below. The components required to enact the system and method of the present invention include a computer control application and informational signal delivery methods which preferably include a computer screen, at least one speaker (30), and mat (10) for stimulation of sympathetic ganglia.

The computer software application of the system of the present invention is preferably configured to facilitate communication between the mat (10) and the computer. The software application preferably includes a USB driver to facilitate the computer's recognition of, and ability to communicate with, the mat (10) of the present invention. It is envisioned that the computer is configured to communicate with only a single iteration of the mat (10) of the present invention. Additionally, the software of the present invention does not require the use of a user management system, nor is a login or password protection required. At least one database, preferably in communication with a computer server is required for data storage.

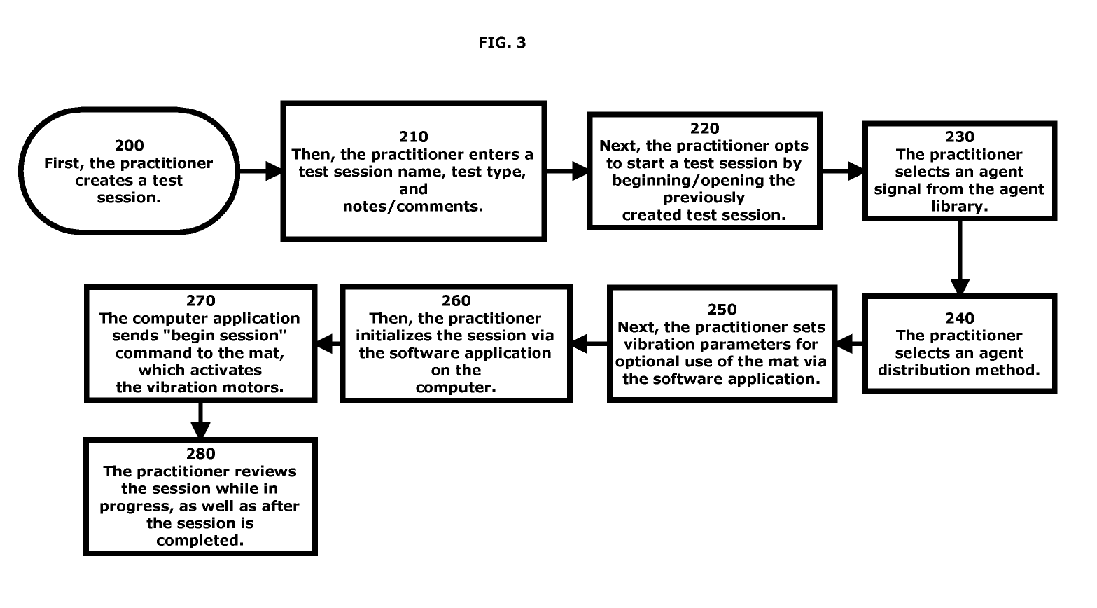

The process of use of the software application component of the system and method of the present invention, as depicted in FIG. 3, is preferably as follows:

### Session/Test Management:

1. First, the practitioner creates a test session. (200)
2. Then, the practitioner enters a test session name (text input), test type (text input), and notes/comments (text input). (210)
3. Next, the practitioner opts to start a test session by beginning/opening the previously created test session. (220)
4. The practitioner selects an agent signal from the agent library. (230) This agent may be presented to the patient in one of several ways.
5. The practitioner selects an agent distribution method. (240) These methods include, but are not limited to:
   Cutaneous exposure of the actual agent
   Audible via Computer
   Visual via Computer
   Audible via Computer and Visual via Computer
   Audible via speakers embedded within the mat (10)
   Audible via speakers embedded within the mat (10) and Visual via Computer
6. Next, the practitioner sets vibration parameters for optional use of the mat via the software application. (250)
7. Then, the practitioner initializes the session via the software application on the computer. (260)
8. The computer application sends “begin session” command to the mat, which activates the vibration motors. (270)
9. The practitioner reviews the session while in progress, as well as after the session is completed. (280)

The process of reviewing previously conducted sessions via the software application of the present invention is preferably as follows:
1. First, the practitioner initiates a ‘Search for sessions.’
2. Next, the practitioner selects a session to open and view.
3. Upon selection, the session is opened, and session data is shown on a display of the computer. The practitioner may opt to save the data as a .CSV file. Similarly, the practitioner may opt to print the results of the session to a .PDF file or to paper. Results may be filtered or displayed by date, by session name, and/or by test type.

### Use of the Sympathetic Chain Stimulation Mat

The use of the method of the present invention preferably employs a mat (10) which provides sympathetic chain stimulation and audible transmission of the agent sounds if the selected mode of stimulus is auditory signals via at least one speaker (30). The mat (10) may be used instead of the aforementioned cuff. During use of the method and system of the present invention, the patient will lie on the mat (10) such that his/her back is in contact with the mat (10). The process of use is preferably the same as described above with the addition of the stimulation functionality listed below.

1. The mat (10) employed in the method of the present invention is preferably 175 cm (68.89 in) long and 62 cm (24.40 in) wide.
2. The mat (10) is preferably equipped with one-inch thick (2.54 cm) foam padding and a cotton covering for patient comfort (see FIG. 2). Additional pillows or towels may be used to provide the patient maximum comfort and head support.
3. The mat (10) is connected to the computer via USB connection.
4. The mat (10) is preferably equipped with an external power supply configured to power the vibration motors (20).
5. The mat (10) is controlled per selections made by the practitioner in the computer software application.
6. The mat (10) has at least one speaker (30) embedded at the upper portion of the mat (10) for audible agent signal transmission. The at least one speaker (30) is embedded within the foam on the upper portion of the mat (near the head) on the side where control wiring enters the mat.
7. The mat (10) has two rows of vibration motors (20) embedded within.
8. Vibration motors (20) are preferably positioned within the mat (10) as follows:
Beginning 35 cm (13.77 in) from the top of the mat
Spaced 3 cm (1.18 in) apart
Extending a total of 60 cm (23.62 in) down the mat towards a bottom of the mat
Final 20 cm (20.87 in) of vibration motors are grouped for separate control to enable flexible use for varying sizes of patients.

A practitioner is able to control the vibration motors (20) via the computer application for the stimulation of the sympathetic ganglia chain. The vibration motors (20) are preferably controllable in four separate groups to target specific regions of the sympathetic chain based on symptoms. Control of the vibration motors (20) is facilitated via the software application. Additionally, the practitioner is able to control the intensity/strength of the vibration motors (20) via the computer software application. Stimulation can be selected to run only for a selected time period during the signal transmission (3-5 seconds), or can be set to run continuously.

It again, should be understood that the stimulation can be administered manually, electrically through TENS, or via the mat (10). Additionally, it should be understood that the present invention is a method of training a living body of a patient, such that the following steps would be performed: administering the offending agent or alternative stimulus modalities to the patient; stimulating the sympathetic chain when administering the stimulus; and storing and matching each signal with the corresponding offending stimulus in a computer database.

Similarly, according to an alternate embodiment of the present invention, when utilizing auditory stimuli, one would place the speaker (30) (which may be embedded into the cuff in this embodiment), near an upper extremity of the patient. Moreover, the present invention can be viewed as positioning a speaker in the proximity of the patient; playing a signal from the speaker toward the patient, the signal matched with a corresponding offending stimulus; and stimulating the entire sympathetic chain locations while playing the signal from the speaker.

Furthermore, the present invention should be viewed as positioning a speaker in the proximity of the patient; be it in within the mat (10) or a cuff, playing, via a computer, signals from the speaker (30) in the proximity of the patient's ears, each signal matched with a corresponding stimulus; facing the speaker toward the body of the patient; converting each signal into a digital format via a computer; storing and matching each signal with the corresponding stimulus in a computer database; placing the speaker onto a cuff or matt, the cuff or matt configured to play the signal in the proximity of the patient's ears. It should be noted that stimulation may be administered manually, electrically or via the mat. The same stimulation procedure may be used when applying the actual offending agent to the surface of the skin while the patient is lying on the mat and stimulating the entire region of the sympathetic chain.

It should be understood that the following are features of the present invention:
Non-invasive, mechanical stimulation of the Sympathetic Ganglia via stimulation of the entire sympathetic ganglia chain (T1-L2) for 5-10 seconds during presentation of conditioned stimulus.
Motor vibration administered via vibration motors (20) disposed within the mat (10) of the present invention.
Manual percussion (any electrical or non-electrical device that elicits a mechanical stimulation).
Transcutaneous electrical nerve stimulation (TENS) is used in preferred embodiments of the present invention for non-responsive cases.
   Low frequency (100 Hz/200 μs) applied with self-adhesive electrodes in the paravertrebral ganglionar region (T1-L2)
   Current delivered at sensory-level intensity for 5-10 seconds
   Includes any form of stimulation to other regions of the body found to decrease activity of the sympathetic nervous system, such as the auricular branch of the vagus nerve.
Representation of offending agents
   Stimuli presented during stimulation of sympathetic ganglia based on the type of offending stimulus.
Visual representations depicted on the computer monitor.
Auditory linguistic representations conveyed via speaker (30) embedded within cuff or mat (10).
Actual stimulus
   The offending stimulus is presented to the surface of the skin. The stimulus includes any form of stimuli that becomes associated with increased sympathetic nerve activity resulting in a pathological or abnormal reaction. This may include, but is not limited to sensitivities, allergies, anxiety disorders, phobias and unwanted behaviors.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A method of training a living body to not react to benign stimuli, comprising:
presenting an agent to the surface of a skin of the living body;
electrically stimulating the sympathetic ganglia by transcutaneous, electrical nerve stimulation; and
forming a new association with the stimulus and modifying a physiological reaction to the stimulus by said a computer playing a digitized sound, the sound being a digital audio representation of a stimulus the living body is inappropriately reacting to, while said electrically stimulating the sympathetic ganglia by transcutaneous, electrical nerve stimulation.

2. The method of claim 1, further comprising:
providing visual signals while a computer plays a digitized sound, the sound being a digital audio representation of a stimulus to which the living body is inappropriately reacting.

3. The method of claim 2, further comprising:
the computer maintaining a database that breaks stimuli down into components.

4. The method of claim 3, further comprising:
a mat providing subcutaneous sympathetic ganglia chain stimulation and audible transmission of sound via at least one speaker.

5. The method of claim 4, wherein the mat contains vibration motors designed for stimulation.

6. The method of claim 5, wherein the vibration motors are positioned centrally within the mat such that the sympathetic ganglia may easily be stimulated when a back of the living body is placed atop the mat.

7. The method of claim 4, wherein the at least one speaker is embedded within the mat.

8. A method of training a living body to not react to benign stimuli, comprising:
presenting an agent to the surface of a skin of the living body;
playing a sound via at least one speaker, the sound being a digital audio representation of a stimulus to which the living body is inappropriately reacting;
stimulating the sympathetic ganglia; and
the living body forming a new association with the stimulus and modifying a physiological reaction to the stimulus.

9. The method of claim 8, further comprising: stimulating the sympathetic ganglia.

10. The method of claim 9, wherein stimulation of the sympathetic ganglia is mechanically achieved via vibration motors disposed within a mat, the mat disposed on a back of the living body.

11. The method of claim 9, wherein stimulation of the sympathetic ganglia is achieved via transcutaneous electrical nerve stimulation.

12. The method of claim 10, further comprising:
a computer interfacing with the mat via a USB cable;
the computer playing a digital audio representation of the stimulus via at least one speaker;
wherein the at least one speaker is embedded within the mat; and
the computer providing visual signals while playing the digital audio representation.
